(19) United States
(12) Patent Application Publication
(10) Pub. No.: US 2008/0038253 A1
(43) Pub. Date: Feb. 14, 2008

(54) Methods For Altering T Cell Diversity

(76) Inventors: Marilia I. Cascalho, Rochester, MN (US); Jeffrey L. Platt, Rochester, MN (US)

Correspondence Address: FISH & RICHARDSON P.C., PO BOX 1022, MINNEAPOLIS, MN, 55440-1022, US

(21) Appl. No.: 10/591,212

(22) PCT Filed: Mar. 4, 2005
(86) PCT No.: PCT/US05/07101

§ 371 Date: Jul. 3, 2007

Related U.S. Application Data
(60) Provisional application No. 60/550,109, filed on Mar. 4, 2004.

(51) Int. Cl. A61K 39/395; A61K 35/12; A61P 43/00

(52) U.S. Cl. 424/133.1; 424/130.1; 424/93.7

(57) ABSTRACT

Methods and materials for enhancing T cell diversity are described. For example, methods that include administering purified populations of B cells or immunoglobulins to subjects in need thereof are provided.

### STATEMENT AS TO FEDERALLY SPONSORED RESEARCH

Funding for the work described herein was provided in part by the federal government, National Institutes of Health (AI48602, AI41570 and HL46810). The federal government may have certain rights in the invention.

### TECHNICAL FIELD

This documents relates to methods for altering the diversity of T cells in subjects, and more particularly, to using B cells or immunoglobulin to alter T cell diversity in subjects.

### BACKGROUND

The generation of T cell receptor diversity is initiated by recombination of the variable (V), diversity (D), and joining (J) gene segments and originates with the variable region of the T cell receptor (TCR) genes in T cell precursors in the thymus. While V(D)J recombination generates billions of different TCRs, only a small fraction of these (5%) is expressed by the mature thymocytes. Thymocytes that fail to produce TCR or that produce TCR that fails to recognize MHC bearing self-peptide die by neglect. Thymocytes bearing self-reactive TCR are eliminated (negative selection), leaving a small fraction of thymocytes surviving (positive selection). Thus, positive and negative selection give rise to a primary T cell repertoire that recognizes self-MHC (restriction) with moderate avidity that is not self-reactive, and in turn establishes the diversity of naïve T cells. Therefore, assuming an equal contribution of V(D)J recombination, the diversity of newly made thymocytes reflects the efficiency of selection. T cell diversity has been estimated to be approximately 10⁸ different T cells in humans and 10⁶ in mice.

### SUMMARY

This description is based, in part, on the findings that diverse B cells and polyclonal immunoglobulin can drive the selection of a diverse repertoire of T cells. As described herein, mice that lack B cells (JH−/−) or that have very few B cells (μMT) have reduced T cell receptor diversity, decreased number of thymocytes, and increased cell death in the thymic cortex. In addition, mice with quasi-monoclonal B cells that are one thousand-fold less diverse than wild type B cells and that have normal numbers of T cells, have severely reduced T cell diversity, indicating that diversity of B cells may be more important than the number of B cells in the generation of a diverse T cell repertoire.

In one aspect, this document features a method for increasing T cell diversity in a subject (e.g., a human subject) in need thereof. The method includes administering a polyclonal population of B cells to the subject. The subject can have an autoimmune disease (e.g., an autoimmune disease selected from the group consisting of rheumatoid arthritis, insulin-dependent diabetes mellitus, myasthenia gravis, systemic lupus erythematosus, and inflammatory bowel disease). The subject also can have AIDS, a congenital immunodeficiency such as severe combined immunodeficiency, common variable immunodeficiency, DiGeorge syndrome, or hyper IgM syndrome, cancer, or a chronic infection. The subject also can have undergone partial or complete thymectomy. The subject can be at least 20 years old.

The method further can include monitoring T cell diversity in the subject. For example, T cell diversity can be monitored using a population of random or diverse nucleic acid molecules.

In another aspect, this document features a method for increasing T cell diversity in a subject in need thereof. The method includes administering polyclonal immunoglobulin to the subject and monitoring T cell diversity in the subject (e.g., a subject as described herein). The polyclonal immunoglobulin can be Fab fragments, reduced monomers, or recombinant. T cell diversity can be monitored using a population of random or diverse nucleic acid molecules.

In yet another aspect, this document features a method for enhancing T cell diversity in a thymectomized subject. The method can include administering polyclonal immunoglobulin to the subject.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 contains graphs of thymocyte numbers in C57BL/6, μMT, and JH−/− mice. The numbers of thymocytes corresponding to each sub-population were calculated by multiplying the respective percentage of the total events as defined in the flow cytometry dot plot analysis, by the total number of WBC obtained by counting on a Coulter. The number of thymocytes (average±SD) was 1.3×10⁸±5.1×10⁷ in C57BL/6, 8.9×10 ⁷±6.4×10⁷ in μMT, and 3.1×10⁷±1.7×10⁷ in JH−/− mice. The average number of CD4^{- CD}8⁻ thymocytes was 5.5×10⁶±2.1×10⁶ in C57BL/6, 3×10⁶±2×10⁶ in μMT, and 2.2×10⁶±2.7×10⁶ in JH−/− mice. The average number of CD4⁺CD8⁺ thymocytes was 9×10⁷±4.4×10⁷ in C57BL/6, 7.3×10⁷±4.7×10⁷in μMT, and 2.3×10⁷±2.7×10⁷ in JH−/− mice. The average number of CD4⁻CD8⁺ thymocytes was 1.2×10⁷±1.2×10⁷ in C57BL/6, 7.7×10⁶±1.3×10⁷ in μMT and 1.5×10⁶±1.4×10⁶ in JH−/− mice. The average number of CD4⁺CD8⁻ thymocytes was 1.9×10⁷±2.8×10⁷ in C57BL/6, 4.9×10⁶±2.8×10⁶ in μMT, and 3.5×10⁶±3×10⁶ in JH−/− mice. Error bars represent standard deviation. Numbers were obtained from 10 C57BL/6, 8 μMT, and 10 JH−/− mice for total number of thymocytes and from 8 C57BL/6, 8 μMT, and 7 JH−/− mice for thymocyte sub-populations. Mice were between 6 and 16 weeks of age. Comparisons of the number of cells in the three strains of mice (indicated by “global p”) were performed by the Kruskal-Wallis test, and comparisons between each two groups of mice (p values indicated below the diagrams) were performed by the Wilcoxon rank sum test.

FIGS. 2A-2D are plots of BrdU incorporation by peripheral blood CD4⁺CD62L⁺ (naïve) T cells, in A and B; or CD8⁺CD62L⁺ T cells, in C and D. x-axis, BrdU staining fluorescence intensity; y-axis, CD62L staining fluorescence intensity. The recent thymic emigrants are the naïve CD4⁺ or CD8⁺ lymphocytes that incorporate low levels of BrdU. The rarity of CD4⁺CD6⁺ and CD8⁺CD6⁺ T cells incorporating low levels of BrdU in thymectomized mice defined the recent thymic emigrants gates (B and D). The dot plots shown are representative of 3 independent experiments with C57BL/6 and JH−/− mice and 2 with μMT mice.

FIG. 3 contains histograms of the DNA content of thymocytes from C57BL/6, μMT, and JH−/− mice. Histograms of one representative experiment of a total of 4 per genotype, are shown. x-axis, DNA content; y-axis, number of cells. Diagrams depict the number of cells in G₁, S and G₂/M phase of the cell cycle. The fraction of cells in S+G₂/M of the cell cycle and the SD were 23%±0.12 for C57BL/6, 19%±0.14 for μMT, and 35%±0.19 for JH−/− mice. 20,000 events were collected for each analysis.

FIG. 4 is a plot of TCR Vβ diversity of thymocytes obtained from C57BL/6, μMT, and JH−/− mice. x-axis, mice strains; y-axis, TCR Vβ diversity. Each open circle represents one experiment, and the values indicate the mean of TCR Vβ diversity for each strain of mice. Statistical analysis was performed on each of the log transformed numeric values by unpaired two sided t test.

FIG. 5A is a flow cytometric analysis of mature B cells in the thymi of C57BL/6, QM, μMT, and JH−/− mice. x-axis, IgM staining fluorescence intensity; y-axis, B220 staining fluorescence intensity. The dot plot diagrams identify mature thymic B cells that are B220⁺ and IgM⁺. Mature B cells are missing from the thymi of μMT and JH−/− mice, but present in the thymi of C57BL/6 and QM mice. Percentages represent the proportion of thymocytes that are mature B cells. Results shown are representative of 3 mice per genotype that were between 6 and 10 weeks of age.

FIG. 5B is a flow cytometric analysis of pro- and pre-B cells in the thymi or bone marrow of C57BL/6, QM, μMT, and JH−/− mice. x-axis, CD43 staining fluorescence intensity; y-axis, B220 staining fluorescence intensity. The plots represent IgM⁻ cells. Upper diagrams represent thymocytes; lower diagrams, bone marrow cells. There were no significant differences in the proportions of pre-B (IgM⁻CD43⁻B220⁺) and pro-B cells (IgM-CD43⁺B220⁺) in JH−/−, μMT, QM, or C57BL/6 thymi. The percentages refer to the fraction of IgM⁻ cells that corresponds to the indicated phenotype. Pre-B cells were nearly absent in the thymi of mice of all genotypes while present in the bone marrow.

FIG. 6A is a graph plotting the TCR Vβ diversity (y-axis) of thymocytes obtained from JH−/− mice injected with polyclonal Ig (with Ig) or polyclonal reduced Ig (with reduced Ig). The controls were JH−/− mice not injected with Ig or reduced Ig (without Ig). Each dot represents a result from one mouse, while the bars represent the average.

FIG. 6B is a graph plotting the TCR Vβ diversity (y-axis) of splenocytes obtained from JH−/− mice injected with polyclonal Ig (with Ig) or polyclonal reduced Ig (with reduced Ig). The controls were JH−/− mice not injected with Ig or reduced Ig (without Ig). Each dot represents a result from one mouse, while the bars represent the average.

FIG. 6C is a graph plotting the TCR Vβ diversity (y-axis) of splenocytes obtained from QM mice injected with polyclonal Ig (with Ig), polyclonal reduced Ig (with reduced Ig), or polyclonal Fab (with Fab). The controls were QM mice not injected with Ig, reduced Ig, or Fab (without Ig). Each dot represents a result from one mouse, while the bars represent the average.

FIG. 7 is a graph plotting the percent of naïve CD4 T cells (CD4-positive, CD62-positive T cells) that are RTE (BrdU intermediate) (y-axis) before and one week after hemi-thymectomy (x-axis). The continuous line represents the fraction of CD4-positive RTE in the blood of mice that received polyclonal Ig. The discontinuous line represents the percentage of CD4-positive RTE in the blood of mice not treated with polyclonal Ig.

### DETAILED DESCRIPTION

In general, this document provides methods and materials for altering cell-mediated immunity in subjects in need thereof using purified populations of B cells or immunoglobulins. Without being bound to any particular mechanism, polyclonal B cells or immunoglobulins provided to a subject can positively select for a diverse range of T cells and can enhance the diversity of the T cell repertoire and increase T cell number, which, in turn, can improve cell-mediated immunity. These findings reveal a heretofore unrecognized and vital function of B cells and immunoglobulin in promoting TCR diversification.

### Polyclonal B cells

The term “purified” as used herein with respect to a population of polyclonal B cells means that the population of cells contains at least 70% B cells (e.g., 80, 90, 95, or 99% B cells). The term “polyclonal” as used herein with respect to a population of B cells means that the population is diverse, i.e., at least 10³ different B cell clones. For example, a polyclonal population of B cells can contain 10⁴, 10⁵, 10⁶, 10⁷, 10⁸, or 10⁹ different B cell clones. Purified populations of B cells can be obtained from peripheral blood, bone marrow, or lymphoid tissue (e.g., lymph nodes or spleen) using standard techniques.

### Polyclonal Immunoglobulin

As used herein, the term “immunoglobulin” refers to intact molecules, including various isotypes, as well as fragments thereof (e.g., Fab, F(ab′)₂, Fv, and single chain Fv (scFv) fragments), and polyclonal or monoclonal immunoglobulins. Immunoglobulin fragments can be generated by known techniques. F(ab′)₂ fragments can be produced by pepsin digestion of antibody molecules. Fab fragments can be generated by reducing the disulfide bridges of F(ab′)₂ fragments. Fab fragments can be generated by treating antibody molecules with papain and a reducing agent. See, e.g., National Institutes of Health, 1 Current Protocols In Immunology, Coligan et al., ed. 2.8, 2.10 (Wiley Interscience, 1991). Fv are antibody products in which there are few or no constant region amino acid residues. An scFv fragment is a single polypeptide chain that includes both the heavy and light chain variable regions of the antibody from which the ScFv is derived. Such fragments can be produced, for example, as described in U.S. Pat. No. 4,642,334.

Gamma globulin, which is collected from pooled plasma of healthy donors and contains IgG immunoglobulins of unknown specificity, is particularly useful in methods provided herein. Gamma globulin is commercially available from, for example, the American Red Cross, Alpha Therapeutic Corporation, and Bayer Corporation. See, also, U.S. Pat. Nos. 4,165,370; 4,465,670; 4,719,290; and 5,164,487.

### Methods of Altering T Cell Diversity

A purified population of polyclonal B cells or polyclonal immunoglobulin can be administered to a mammal such as a human patient having a deficiency in cell-mediated immunity. Typically, the polyclonal B cells are autologous to the mammal. In cases in which a polyclonal population of B cells cannot be harvested from a mammal, polyclonal immunoglobulin can be administered.

Deficiencies in cell-mediated immunity may arise, for example, in subjects who have undergone partial or complete thymectomy or in subjects with weakened immune systems due to AIDS, congenital immunodeficiencies (e.g., severe combined immunodeficiency, common variable immunodeficiency, DiGeorge syndrome, or hyper IgM syndrome), organ transplants, cancer, chronic infections, or aging (e.g., patients 20 or more years of age). Autoimmune diseases such as rheumatoid arthritis, insulin-dependent diabetes mellitus, myasthenia gravis, systemic lupus erythematosus, and inflammatory bowel disease also can result in deficiencies in cell-mediated immunity. Purified populations of polyclonal B cells or polyclonal immunoglobulin also can be administered prophylactically in patients at risk for developing a deficiency in cell-mediated immunity to, for example, prevent development of or lessen the severity of the deficiency in cell-mediated immunity.

In either case, an amount of a purified population of polyclonal B cells or polyclonal immunoglobulin effective to enhance T cell diversity can be administered to the subject. As used herein, the term “effective amount” refers to an amount that enhances the diversity of the subject's T cells without inducing significant toxicity to the host. Effective amounts of a purified population of polyclonal B cells or polyclonal immunoglobulin can be determined by a physician, taking into account various factors, including overall health status, body weight, sex, diet, time and route of administration, other medications, and any other relevant clinical factors. Typically, at least one million B cells, e.g., 10⁶, 10⁷, 10⁸, or more cells, or ≧1 mg of immunoglobulin can be administered.

B cells or immunoglobulin can be administered by many different routes, including, without limitation, oral or parenteral routes of administration such as intravenous, intramuscular, intraperitoneal, subcutaneous, intraarterial, nasal, transdermal (e.g., as a patch), or pulmonary absorption. In some embodiments, the cells or immunoglobulins can be implanted, e.g., into the thymus. Purified populations of polyclonal B cells or polyclonal immunoglobulin can be formulated as, for example, a solution, suspension, or emulsion with pharmaceutically acceptable carriers or excipients suitable for the particular route of administration, including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives, flavorings, sugars, and other additives such as antimicrobials, antioxidants, chelating agents, inert gases, and the like also may be present.

In some embodiments, monoclonal B cells or immunoglobulin are administered to a subject. For example, monoclonal cells or immunoglobulin can be administered to a patient having a tumor to increase the ability of the patient's immune system to kill the tumor cells.

### Monitoring T cell Diversity

T cell diversity can be monitored in a subject to determine if T cell diversity has been altered relative to the subject's baseline level of diversity (i.e., level of TCR diversity before administration of B cells or immunoglobulin). Indirect or direct methods of assessing T cell diversity can be used. For example, the methods of Ogle et al., 2003, Nucleic Acids Res., 31:e139 can be used to directly assess T cell diversity. See, also, U.S. patent application Ser. No. 60/464,981, filed Apr, 24, 2003. In this method, the number of hybridization spots “hits” of TCR Vβ chain RNA can be determined using a gene chip (e.g., U95B, Affimetrix Inc., Santa Clara, Calif.). The diversity of the TCR Vβchain in a population is proportional to the number of hits above background (defined by the number of hits corresponding to hybridization of monoclonal TCR Vβ chain RNA) of TCR Vβ chain-specific RNAs on the gene chip. Diversity can be calculated by plotting the number of “hits” onto a standard curve obtained for each experiment by hybridizing oligonucleotide mixtures of known diversity to individual gene chips.

Indirect techniques include methods using antibodies against variable (V)-region families and flow cytometric analysis of lymphocyte populations. Sheehan et al., Embo J. 8:2313-20 (1989); Langerak et al., Blood 98:165-73 (2001). This approach can detect “constant” antigenic determinants shared by many lymphocyte receptor clones, and diversity is inferred from the result. Another method of indirect assessment includes amplifying nucleic acids encoding lymphocyte receptors by the polymerase chain reaction (PCR) using constant region (C) and V family specific primers. Murata et al., Arthritis Rheum. 46:2141-7 (2002). Diversity also can be estimated by spectratyping or immunoscope. See, Pannetier et al., Proc. Natl. Acad. Sci. USA 90:4319-23 (1993); Pannetier et al., Immunol. Today 16:176-81 (1995); and Delassus et al., J. Immunol. Methods 184:219-29 (1995). After V specific families are amplified by PCR, a fluorescently labeled junctional region (J) primer can be used for a “run off” PCR reaction; the products of which can be separated on sequencing gels. Amplified lymphocyte receptor families (specified by the primers used in the initial PCR) can migrate in a series of bands, each of which corresponds to a different length of the complementarity determining region 3 (CDR3-TCR region believed to harbor the largest portion of genetic variability). In normal lymphocyte populations, the CDR3 size distribution is Gaussian for each variable region family, and so any alteration in distribution and/or band intensity is attributed to a perturbation of diversity. Another indirect method of monitoring lymphocyte diversity is based on the tenets of limiting dilution analysis and detects the frequency of a given TCR clone. Wagner et al., Pro. Natl. Acad. Sci. USA 95:14447-52 (1998).

### Articles of Manufacture

Purified populations of polyclonal B cells can be combined with packaging materials and sold as articles of manufacture or kits (e.g., for enhancing T cell diversity). Components and methods for producing articles of manufactures are well known. The articles of manufacture may combine one or more components described herein. In addition, the articles of manufacture further may include reagents to monitor T cell diversity such as reagents to label nucleic acid molecules, nucleic acids that can serve as positive or negative controls, and/or reagents for preparing a standard curve. Instructions describing how a purified population of polyclonal B cells can be used to enhance T cell diversity also can be included.

The invention will be further described in the following examples, which do not limit the scope of the invention described in the claims.

### EXAMPLES

### Materials and Methods

Mice. JH−/−, μMT, monoclonal B—T, and quasi-monoclonal (QM) mice have been described (Chen et al., 1993, Int. Immunol., 5:647; Kitamura et al., 1991, Nature, 350:423; Keshavarzi et al., 2003, Scand. J. Immunol., 57:446; and Cascalho et al., 1996, Science, 272:1649). The B cell-deficient strains consisted of the JH−/− mice obtained by gene targeted deletion of the JH segments, and the μMT mice were obtained by gene targeted disruption of the μ immunoglobulin heavy chain membrane exons. C57BL/6 and μMT mice were purchased from the Jackson Laboratories. JH−/−, monoclonal B—T, and QM mice were bred, and all mice were housed in a specific pathogen-free facility at the Mayo Clinic. All mice were between 6 and 16 weeks of age and were age matched. All animal experiments were carried out in accordance with protocols approved by the Mayo Clinic Institutional Animal Care and Use Committee.

Adoptive transfer. Bone marrow cells were harvested from C57BL/6 mice or monoclonal B—T mice, and lymphocytes were isolated by Ficoll-paque gradient (Amersham Biosciences, Piscatanaway, N.J.). Polyclonal B cells (1×10⁷) purified with a Miltenyi Biotec isolation kit and monoclonal B cells (6×10⁶) purified with a high-speed sorter FACSVantage SE, (Becton Dickinson), were injected i.p. in newborn mice (20 μL). The polyclonal B cells contained, on average, 1% of CD3⁺CD4⁺ and 1.5% of CD3⁺CD8⁺ cells, and the monoclonal B cells contained, on average, 0% of CD3⁺CD4⁺ and 0.07% of CD3⁺CD8⁺ cells. Immunoglobulin or ovalbumin injections were done i.p., weekly with 250 μg of mouse polyclonal IgG (Seroteca) or monoclonal anti-KLH IgG2b (C48-4, BD Biosciences), or with 250 μg ovalbumin (Sigma), since birth.

Immunoglobulin injections. JH−/− mice were injected i.p. weekly with 250 μg of mouse polyclonal IgG (Seroteca) or monoclonal anti-KLH IgG2b (C48-4, BD Biosciences), since birth. Serum levels of total immunoglobulin were tested 4 weeks or later after the first injection.

FACS analysis. Thymocytes were obtained by mincing thymi through a 0.70 μm mesh followed by red blood cell hemolysis in a standard NH₄Cl lysis buffer. Bone marrow cells were prepared by flushing femurs with cell suspension buffer followed by red blood cell lysis, as described by Cascalho et al. (1996, Science, 272:1649). Total thymocyte numbers were counted with a Coulter counter. Cells were stained with one, two, or three of the following monoclonal antibodies (all the antibodies were from BD Pharmingen) as described by Cascalho et al. (1996, Science, 272:1649): fluorescein isothyocyanate (FITC)-conjugated rat anti-mouse CD4 (GK 1.5), rat anti-mouse CD43 (Ly-48, leukosialin), and mouse anti-BrdU antibodies; phycoerythrin (PE)-conjugated rat anti-mouse CD8α (53-6.7), rat anti-mouse CD19 (1D3), rat anti-mouse IgM^{b} (Igh-6b)(AF6-78); and biotin-conjugated rat anti mouse B220 (16A), rat anti-mouse CD62L (LECAM-1, Ly22) and rat anti-mouse CD3_{ε} (145-2C11). Lymphocytes were gated on the light scatter plot by backgating onto CD3⁺CD5⁺ and CD8⁺CD3⁺ cells; numbers of the thymocyte sub-populations were determined by multiplying the percentage as defined by gating on the FACS plot, by their total number.

DNA analysis. Thymocytes (10⁶/mL) were washed with ice-cold phosphate buffered saline (PBS, pH=7.2) and fixed in 70% ethanol at −20° C. for at least 2 hours. After fixation, cells were washed twice with PBS and incubated in 50 μL of DNA extraction buffer (0.2 M phosphate citrate buffer, pH=7.8) at 37° C. for 30 min in the shaker. Following DNA extraction, the cells were stained with propidium iodide (PI) in a solution containing 10 ml of 0.1% (v/v) of Triton X-100 in PBS, 200 μL of 1 mg/mL PI (Molecular Probes) and 2 mg of DNase-free Rnase A (Sigma), for 30 min at room temperature. Detection of PI fluorescence was read at red wavelength in a FACScan flow cytometry (Becton Dickinson) and analyzed with the ModFit LT software.

Immunohistological analysis. Thymi removed from six to eight week old mice were oriented and covered with O. C. T (Sakura, Torrance, Calif.), snap-frozen by pre-cooled isopentane and stored at −85° C. Four micron thick frozen sections were mounted on positively-charged microscope slides (Superfrost Plus, Fisher Scientific, Pittsburgh, Pa.) and stored at −85° C. Before processing, sections were air-dried at room temperature, fixed 10 minutes in 4° C. acetone, air-dried for an additional 10 minutes, then post-fixed for two minutes in 100 mM Tris-buffered 1% paraformaldehyde containing 1 mM EDTA, pH 7.2, and rinsed with PBS. Prior to staining, the specimens were incubated in 0.3% hydrogen peroxide in 0.1% sodium azide (aq) solution to quench the presence of endogenous peroxidase. Specimens were incubated with rat monoclonal antibodies to murine CD19 (clone 1D3, BD Pharmingen, San Diego, Calif.) and to CD45R/B220 (clone RA3-6B2, BD Pharmingen), rinsed with PBS, then detected by mouse serum preabsorbed, affinity purified, biotinylated Goat IgG anti-rat IgG (Jackson ImmunoResearch, West Grove, Pa.), followed by PBS rinses and the tertiary application of horseradish-conjugated streptavidin (Dako, Carpinteria, Calif.). Slides were developed by incubation with a peroxidase substrate NovaRED (Vector Laboratories, Burlingame, Calif.), which resulted in an insoluble reddish-brown precipitate, followed by counterstaining with a progressive alum-hematoxylin, dehydrated in graded ethanols, cleared in xylene changes, and cover-slipped with Cytoseal-Xyl (Stephens Scientific, Kalamazoo, Mich.). Digital images were obtained using a brightfield microscope equipped with a CCD digital camera (SPOT II, Diagnostic Instruments, Sterling Heights, Mich.).

TUNEL. Apoptotic cells were detected in cryostat sections of thymi by in situ terminal deoxynucleotidyltransferase-mediated 2′-deoxyuridine 5′-triphosphate nick end-labeling (TUNEL), performed according to the manufacturers instructions (ApopTag™plus Peroxidase kit, Serologicals Corporation).

### Determination of TCRVβ diversity.

Isolation of RNA. Thymi harvested from 5 to 12 week old mice were placed in RPMI and pushed through a 70 μm cell strainer. Lymphocytes were isolated by Ficoll-paque (Amersham Biosciences, Piscatanaway, N.J.) gradient. Total RNA was obtained with Qiagen RNeasy kit (Qiagen, Inc., Valencia, Calif.) per the manufacturer's instructions.

Generation of diversity standards. Diversity standards were prepared by creating oligonucleotide mixtures of known diversity. For example, to generate a standard diversity of 10⁶, 18-mer oligonucleotides were synthesized with 10 sites of random assignment generating 4¹⁰=1,040,526 different oligomers. Similarly, oligomer mixtures were created with 1, 10³, and 10⁹ variants. Oligonucleotides were biotin-labeled and hybridized to the gene chips as explained below.

Generation of lymphocyte receptor-specific cRNA. First strand cDNA was obtained by reverse transcription with a mouse TCR Cβ reverse primer: T7+Cβ (5′-GGCCAGTG

AATTGTAATACGACTCACTATAGGGAGGCGGCTTGGGTGGAGTCACATTTCTC −3′, SEQ ID NO:1) with SuperScript II Reverse Transcriptase (Invitrogen Corporation, Carlsbad, Calif.). This primer contains a T7 polymerase promoter 3′ overhang annealed to the TCR β constant region. Second strand synthesis (nick translation) and preparation of biotin-labeled cRNA was conducted according to Affymetrix standard protocols (Affymetrix, Inc., Santa Clara, Calif.).

Application of cRNA to the gene chip. Equal amounts of cRNA from different samples and diversity standards were hybridized to U95B gene chips (Affymetrix, Inc., Santa Clara, Calif.). Gene chips were processed at the Microarray Core Facility, Mayo Clinic, Rochester, Minn.

Data analysis. For each gene chip experiment, raw data were obtained corresponding to oligo location and hybridization intensity. Data were arranged in order of ascending hybridization intensity. The number of oligo locations with intensity above background (i.e., number of hits) was summed. The standard curve was generated from hybridization of samples with known numbers of different oligomers. The standard oligonucleotide mixtures were 18-mer oligomers synthesized to obtain mixtures containing 1, 10³, 10⁶, and 10⁹ different oligonucleotides. Diversity of cRNA obtained from monoclonal T cells was used to establish the background and diversity of the test samples was extrapolated directly from the standard curve. TCR specificity of the Cβ reverse primer was controlled for by determining the diversity of cRNA obtained from purified polyclonal B cells (with less than 0.9% of T cells) with the Cβ reverse primer, which was found to be 3 per 10 μg of RNA and indistinguishable from background.

Statistical analysis. Statistical analysis for group comparison of means of TCR Vβ diversity of thymocytes was performed using log transformation of the data followed by one-way Anova analysis of variance. Groups of two comparisons were performed by unpaired, two-sided Student's t test. Comparisons of thymocyte numbers were performed using the Kruskal-Wallis test for global differences followed by the Wilcoxon rank sum test. A p value<0.05 was considered to be statistically significant.

### EXAMPLE 1

### Thymocyte Development is Perturbed in Mice That Lack B Cells and Immunoglobulin

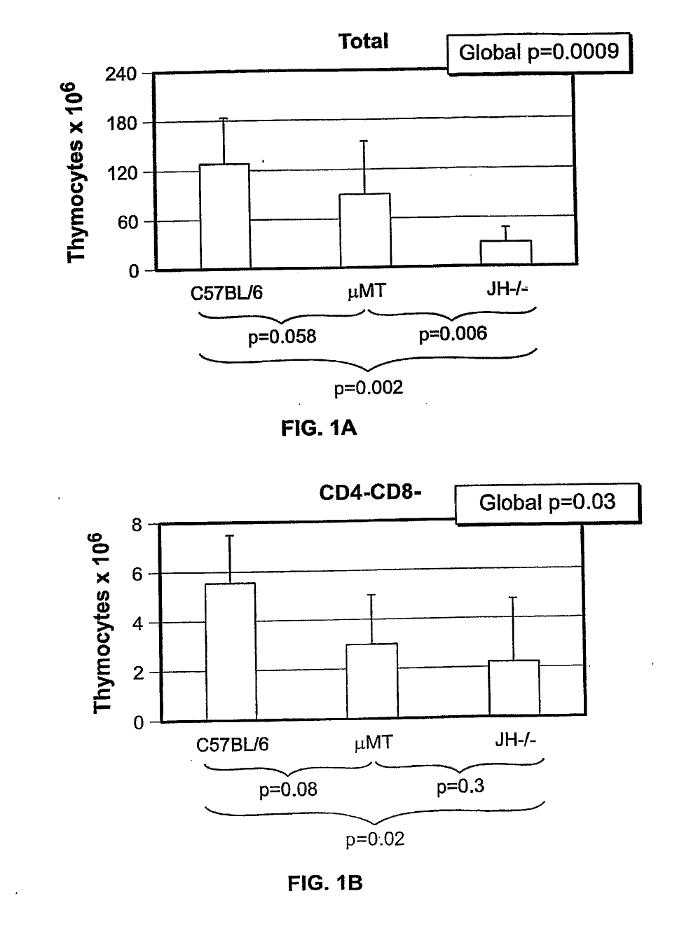

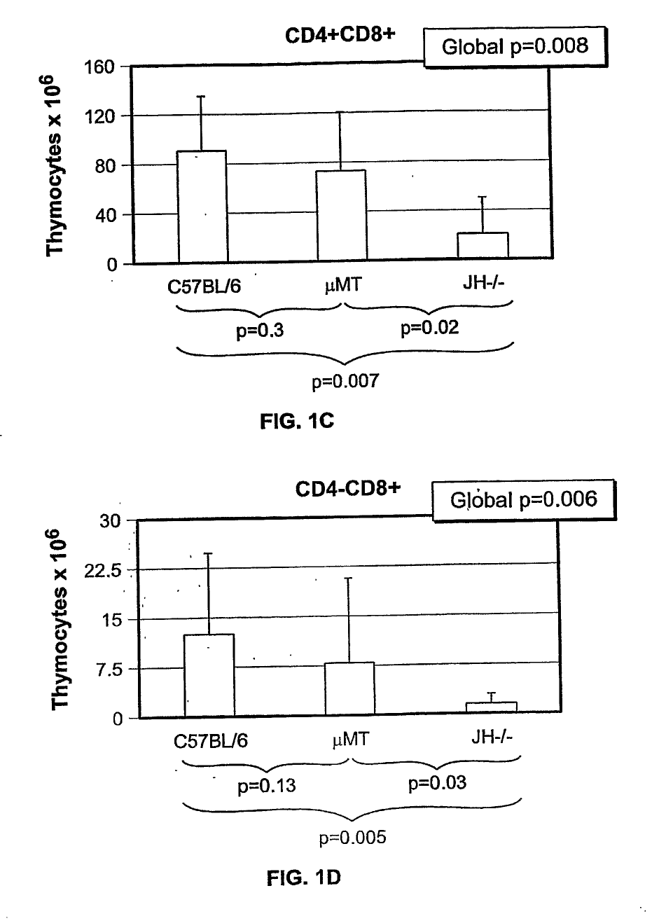

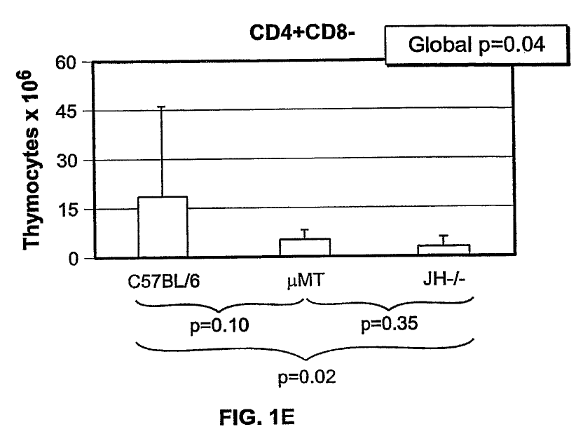

To test whether B cells and/or immunoglobulin might contribute to thymic selection, the numbers of thymocytes and thymocyte sub-populations were compared in mice that lack B cells and immunoglobulin (JH−/−) with the numbers in wild type mice (C57BL/6). Because the thymus atrophies with age, the mice in each group were age matched. The results show that JH−/− mice had significantly fewer total thymocytes (6.5-fold) compared with C57BL/6 (FIG. 1). The smaller number of thymocytes in JH−/− mice was mainly owed to a 3.9-fold decrease in the number of CD4⁺CD8⁺ thymocytes (FIG. 1), but also reflected a significant decrease in the numbers of CD4⁻CD8⁻, CD4⁺CD⁸⁻, and CD4⁻CD8⁺ populations compared with wild type. These results suggest that B cells and/or a B cell product such as immunoglobulin might influence thymocyte development by various direct or indirect means.

To find whether immunoglobulin might on its own contribute to T cell development, thymocytes were examined in the μMT mouse that has serum immunoglobulin in a concentration of 4.5% of wild type but very few B cells (Macpherson et al., 2001, Nat. Immunol. 2:625) (Table I). The number of thymocytes in μMT mice was increased significantly by 3.2 fold compared with JH−/− mice (FIG. 1). These results are consistent with a role for immunoglobulin in the development of T cells.

### EXAMPLE 2

### Increased Apoptosis in the Thymic Cortex of Mice That Lack B Cells and Immunoglobulin

In this experiment, it was determined whether the fewer thymocytes in JH−/− mice were the result of higher levels of cell death. Consistent with that concept, TUNEL, which detects DNA strand breaks in cells undergoing apoptosis, revealed increased apoptosis in the thymic cortex of JH−/− mice compared to μMT or to C57BL/6 mice. The number of apoptotic spots in JH−/− sections was at least 2.5 fold greater than the number of spots counted in equivalent areas of C57BL/6 or μMT sections. Increased cell death could be the consequence of decreased positive selection and/or increased negative selection. Because defective positive selection is accompanied by cortical thymocyte apoptosis (Surh and Sprent, 1994, Nature, 372:100), these results are compatible with B cells and/or immunoglobulin promoting thymic positive selection. Since apoptosis in the thymic cortex of μMT mice is comparable to apoptosis detected in C57BL/6 mice and since μMT mice produce serum immunoglobulin but few B cells, these results suggest that serum immunoglobulin promotes thymocyte survival.

### EXAMPLE 3

### The Numbers of Recent Thymic Emigrants are Maintained and Thymocyte Proliferation is Increased in Mice That Lack B Cells and Immunoglobulin

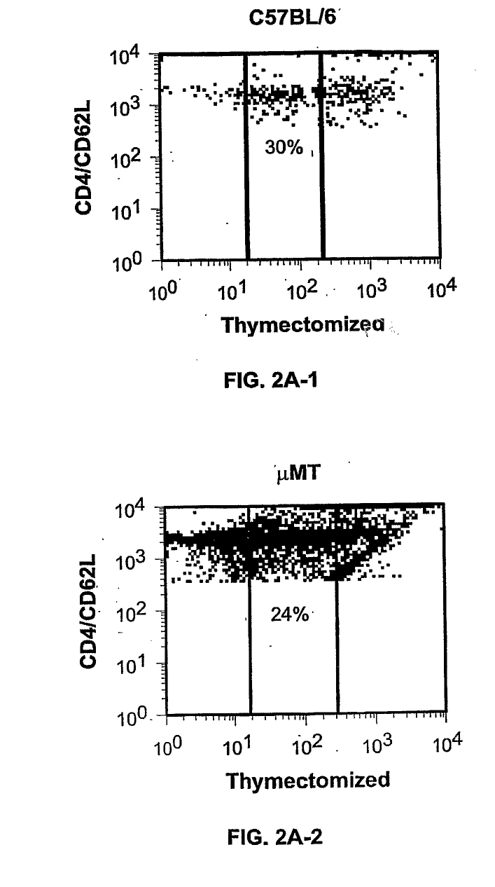

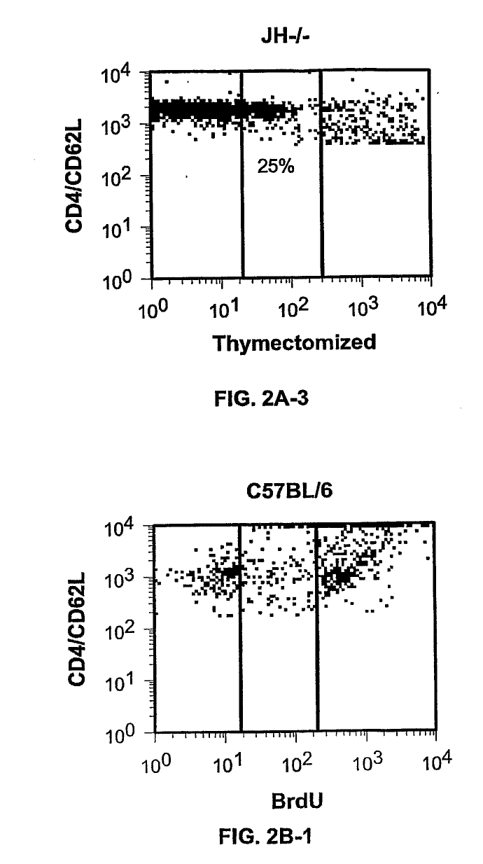

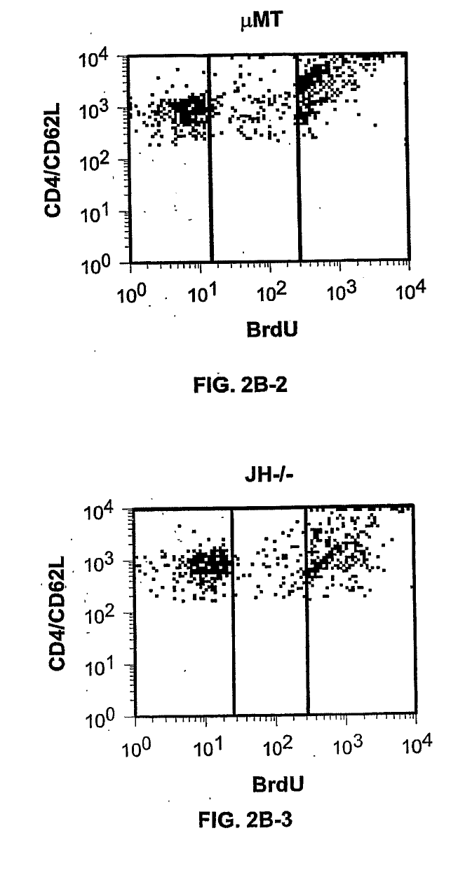

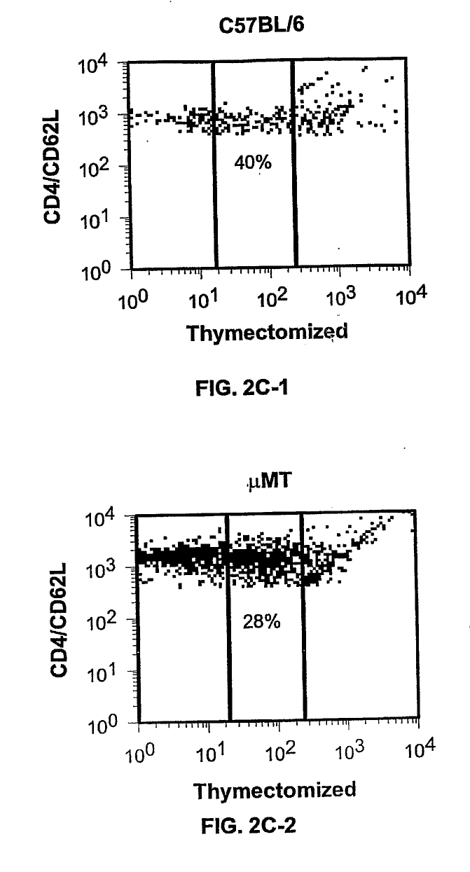

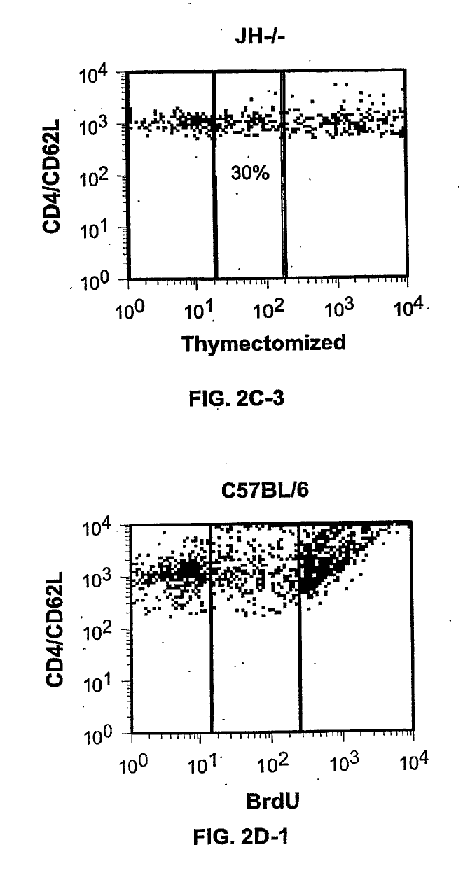

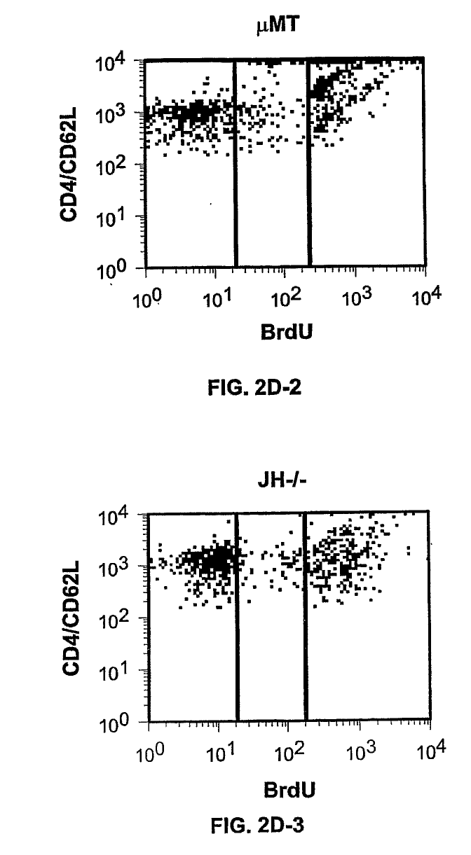

This experiment determined whether decreased numbers and increased apoptosis of thymocytes in JH−/− mice would change the numbers of recent thymic emigrants. Recent thymic emigrants were identified based on their tendency to take up relatively low levels of BrdU according to the method of Tough and Sprent (1996, Curr. Prot. Immunol. Suppl. 18:4.7.1; Sprent and Tough, 1994, Science, 265:1395). Recent thymic emigrants gate was defined by comparing thymectomized and non-thymectomized mice treated with BrdU. The recent thymic emigrants gate includes the population of naïve T cells lost by thymectomy (FIGS. 2B and 2D). Using these gates, no differences were found in the proportions of recent thymic emigrants in CD4⁺ or CD8⁺ naïve (CD62L-positive) T cells analyzed in JH−/−, μMT, and C57BL/6 mice, suggesting that thymic output is maintained despite decreased number of thymocytes (FIGS. 2A and 2C).

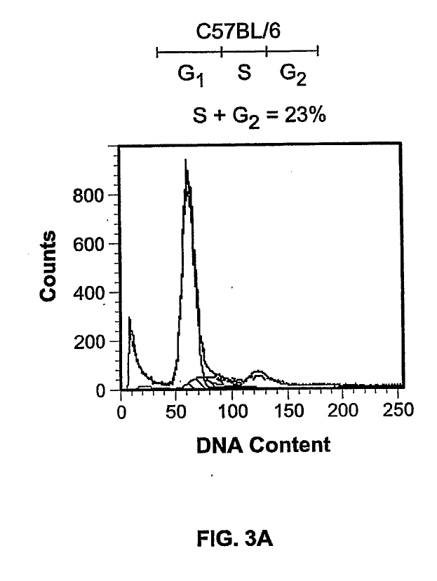

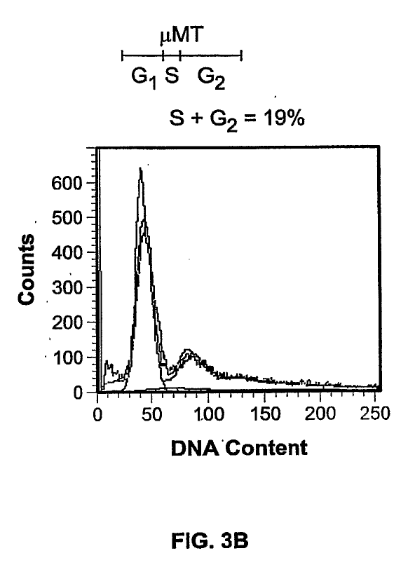

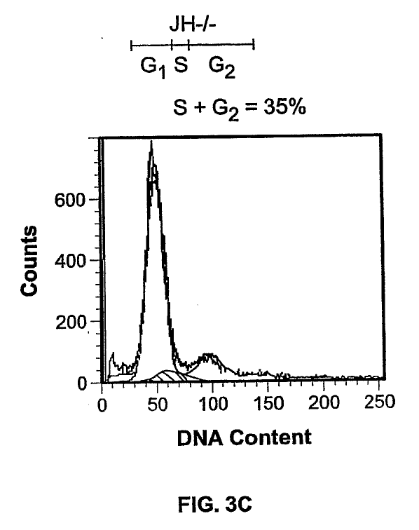

To reconcile the decreased number of thymocytes in JH−/− mice with the maintenance of thymic output, it was hypothesized that these mice have increased proliferation of thymocytes. Increased thymocyte proliferation would allow JH−/− mice to maintain thymic output despite increased cell death. Cell cycle analysis of thymocytes of JH−/−, μMT, and C57BL/6 mice revealed a 1.5 fold increase in the number of cycling (S+G₂/M) thymocytes in JH−/− compared to μMT and C57BL/6 mice (FIG. 3). These results suggest that lack of B cells and immunoglobulin leads to increased cell death and to thymocyte proliferation, perhaps as a compensatory mechanism to maintain the T cell production.

### EXAMPLE 4

### Contraction of the T Cell Receptor Repertoire in Mice That Lack B Cells and or Immunoglobulin

If mice that lack B cells and immunoglobulin have fewer thymocytes and a normal T cell egress, one might predict that the TCR repertoire would be contracted, owing to clonal expansion of the fewer surviving thymocytes. As a direct test of this idea, TCR diversity was assayed in JH−/−, μMT, and C57BL/6 mice.

To measure TCR diversity directly, the number of hybridization spots “hits” of TCR Vβ chain RNA was determined on a gene chip (U95B, Affimetrix Inc., Santa Clara, Calif.) as described above. See, Ogle et al., 2003, Nucleic Acids Res., 31:el39. The diversity of the TCR Vβ chain in a population is proportional to the number of hits above background (defined by the number of hits corresponding to hybridization of monoclonal TCR Vβ chain RNA) of TCR Vβ chain-specific RNAs on the gene chip. Diversity was calculated by plotting the number of “hits” onto a standard curve obtained for each experiment by hybridizing oligonucleotide mixtures of known diversity to individual gene chips. The number obtained varies proportionally to the actual TCR Vβ diversity even though it does not represent the number of different TCR Vβ chains since each TCR Vβ chain generates more than one hit. To determine whether the TCR Cβ primer cross-hybridized with B cells RNA, the diversity of cRNA obtained from purified B cells was determined with the Cβ reverse primer. It was found that the number of hits obtained from B cells' cRNA prepared with the TCR Cβ primer was 3 per 10 μg of RNA and indistinguishable from background indicating that the TCR Cβ primer did not cross-hybridize with B cells RNA. In addition, T cell diversity of C57BL/6 splenocytes, which include mostly B cells, was 1000 fold lower than T cell diversity of C57BL/6 thymocytes (results not shown). Thus diversity of TCR Cβ specific cRNA does not reflect contamination with B cell receptor.

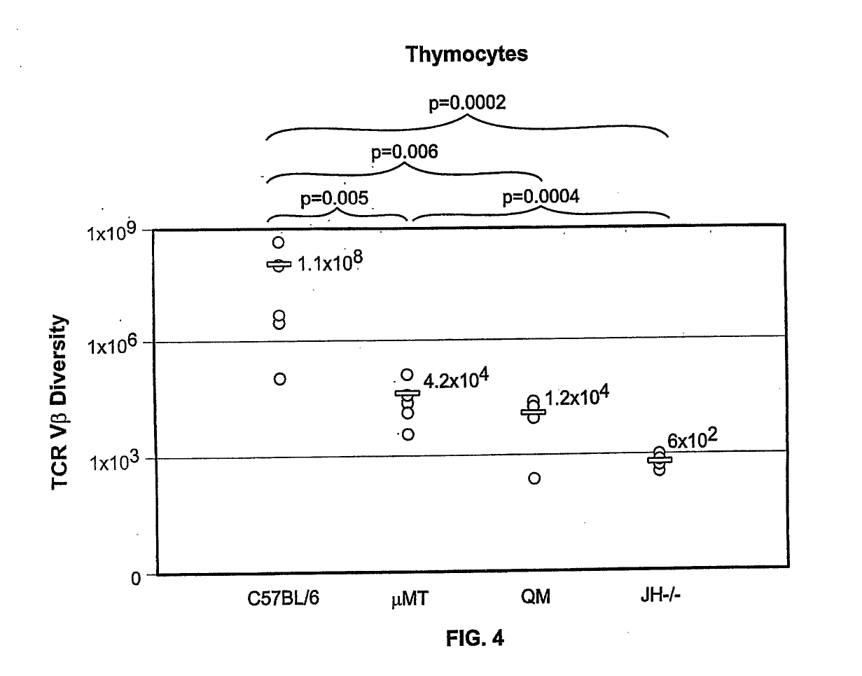

FIG. 4 shows that the TCR Vβ diversity of JH−/− thymocytes was 6.0×10² per 10 μg of RNA compared with wild type that was 1.1×10⁸ per 10 μg of RNA (p=0.0002). By showing profoundly decreased thymocyte diversity in mice that lack B cells and immunoglobulin (JH−/−), the results indicate that B cells and/or immunoglobulin promote thymocyte diversity. In this experiment, age matched JH−/− and C57BL/6 mice were compared that were between 5 and 12 weeks old. Therefore, the decreased thymocyte diversity in JH−/− mice was not due to age dependent atrophy of the thymus.

Next, it was asked whether immunoglobulin could promote T cell diversity under the condition of B cell deficiency. To this end, the TCR Vβ diversity was analyzed in thymocytes of μMT mice that have serum immunoglobulin but very few B cells. It was found that TCR Vβ diversity in thymocytes of μMT mice was 4.2×10⁴ per 10 μg of RNA and 70 fold greater than TCR Vβ diversity in thymocytes of JH−/− age matched mice (6.0×10² per 10 μg of RNA) (p=0.0004). The results indicate that immunoglobulin and/or the few B cells in μMT mice contribute to T cell diversity. The reduced TCR diversity in B cell-deficient mice was maintained in the periphery since JH−/− and μMT mice splenocytes had a 1000 fold and 10 fold reduced TCR Vβ diversity compared to wild type (results not shown).

Selection of a diverse T cell repertoire requires T cell receptor recognition of diverse self-peptides in the context of self-MHC. Because the variable regions of heavy and light chain of antibodies are a potential source of diverse self-peptides, it was determined whether T cell diversity depended on the diversity of the B cells. To address this question, TCR Vβ chain diversity was assessed of thymocytes obtained from quasi-monoclonal (QM) mice that have 80% of the B cells from a single clone and polyclonal serum immunoglobulin. In QM mice, the diversity of the JH₄ containing heavy chains is only 0.01% of wild type. FIG. 4 shows that the TCR Vβ diversity of QM thymocytes was, on average, 1.2×10⁴ per 10 μg of RNA, not significantly different from μMT and 0.01% of wild type thymocyte diversity (p=0.006). These findings of comparable TCR diversity in QM and μMT age matched mice indicate that oligoclonal B cells do not promote diversification of T cells.

### EXAMPLE 5

### B Cell Precursors in C57BL/6, QM, JH−/−, and μMT Thymi

The results described above imply that B cells and/or immunoglobulin promote selection of a diverse T cell repertoire presumably in the thymus. Since both mature B cells and B cell precursors are found in the thymus, it was determined which B cell populations were present in thymi of C57BL/6, QM, JH−/−, and μMT mice. Thymic sections of C57BL/6, μMT, and JH−/− mice were immunohistochemically stained. Positive cells stained brown. JH−/− and μMT thymi had fewer CD 19⁺ cells in the thymic cortex and fewer CD19 ⁺ and B220⁺ cells in the medulla in comparison to C57BL/6 mice. In contrast, the numbers of B220⁺ cells were comparable in the thymic cortices of JH−/−, μMT, and C57BL/6 mice.

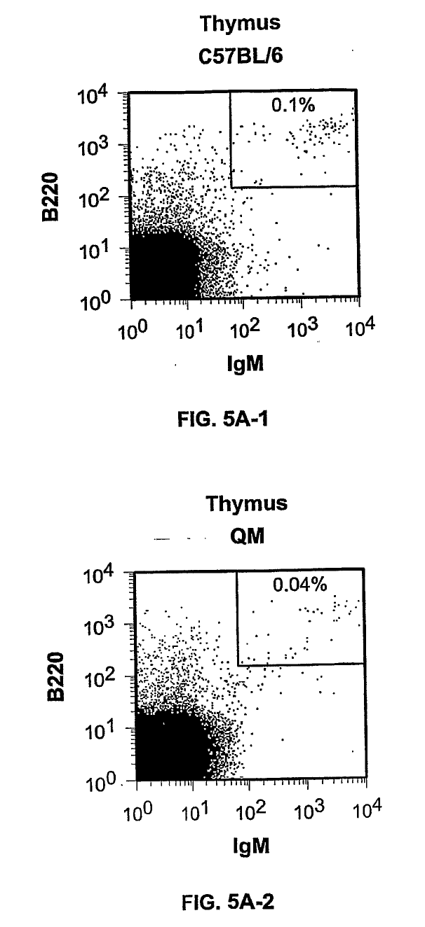

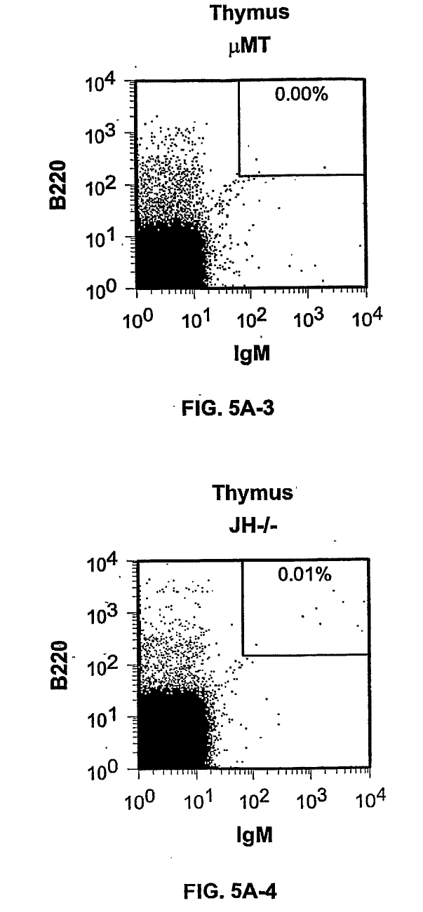

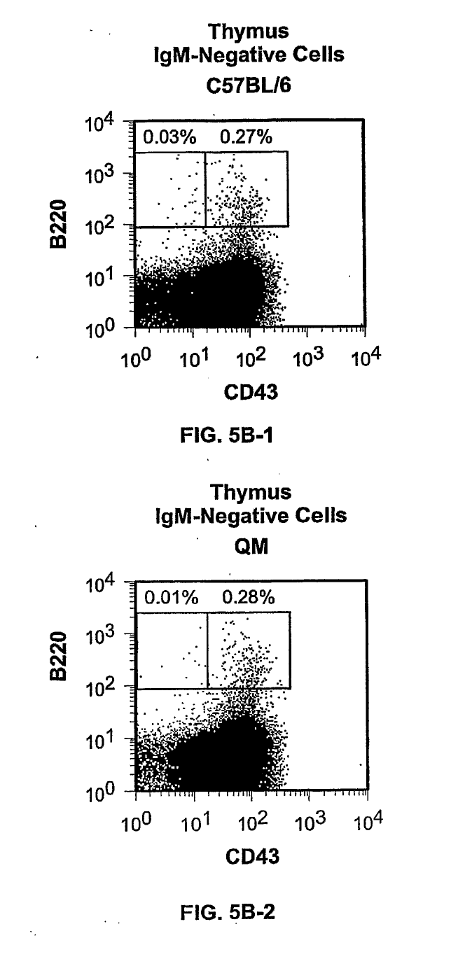

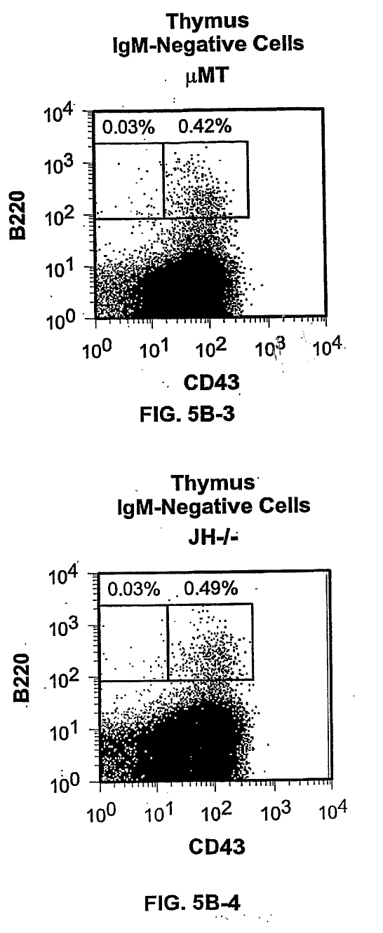

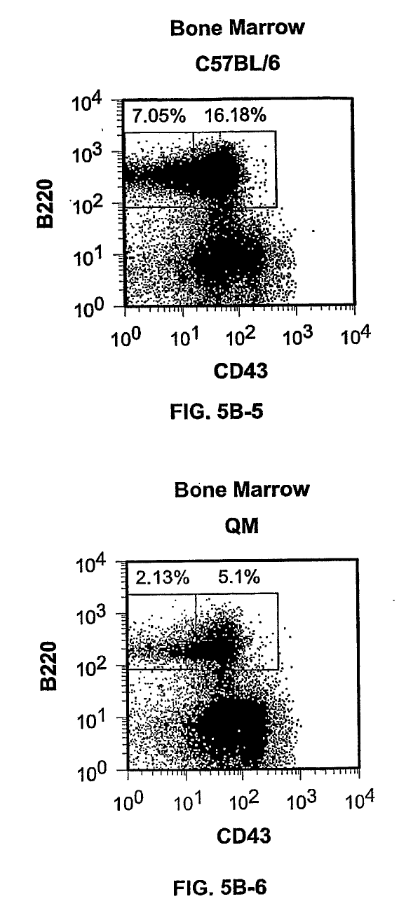

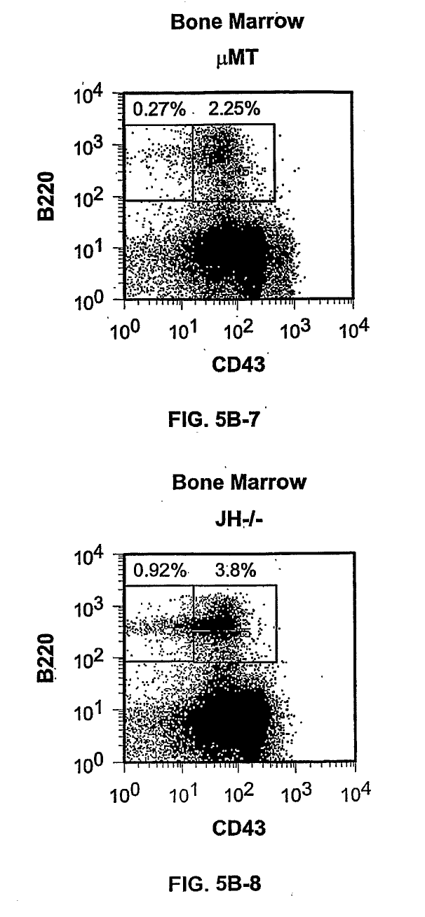

To discriminate B cell precursors from mature B cells, thymocytes of JH−/−, μMT, QM, and C57BL/6 mice were analyzed by flow cytometry. FIG. 5A shows that mature B cells (IgM⁺ and B220⁺) are missing from JH−/− and μMT thymi and reduced by half in QM thymi compared to C57BL/6 thymi. There were very few pre-B cells (IgM⁻CD43⁻B220⁺) (Li et al., 1993, J. Exp. Med. 178:951) in the thymi of mice of all genotypes while they were present in the bone marrow (FIG. 5B). Pro-B cells (IgM⁻CD43⁺B220⁺) did not differ significantly in JH−/−, μMT, QM, or C57BL/6 thymi (FIG. 5B). These findings thus suggest that mature B cells rather than B cell precursors promote thymocyte selection and the generation of T cell diversity.

### EXAMPLE 6

### T Cell Diversity in JH−/− Mice Reconstituted by Adoptive Transfer of B Cells or Administration of Immunoglobulin

Next it was tested whether providing B cells and/or immunoglobulin could increase TCR diversity in JH−/− mice lacking both B cells and immunoglobulin. To this end, newborn JH−/− mice were injected with bone marrow-derived wild type or monoclonal B cells, or with polyclonal or monoclonal IgG, and TCR diversity was measured after 4 weeks. The presence of adoptively transferred B cells was verified by flow cytometry analysis; recipient mice had between 10% and 20% of B cells in peripheral blood lymphocytes, at the time of sacrifice. Mice injected with immunoglobulin had, on average, serum concentrations greater by 4.7 fold than wild type at the time of sacrifice (Table I).

TABLE I
Mean concentration of serum immunoglobulin ± SD in C57BL/6,
QM, mMT, JH−/− mice and in JH−/− mice reconstituted
with B cells or following administration of IgG.

| Mice | Serum Immunoglobulin (μg/ml) |
|---|---|
| C57BL/6 | 163.5 ± 160.6 |
| μMT | 10.7 ± 24.7 |
| QM | 654.5 ± 248.8 |
| JH−/− | 0 |
| JH−/− with Polyclonal B cells | 0 |
| JH−/− with Polyclonal IgG | 832.2 ± 1380.1 |

Transfer of wild type B cells in JH−/− mice increased thymocyte TCR Vβ diversity by 8-fold; however, adoptive transfer of monoclonal B cells did not, indicating that B cell diversity is required for the generation of thymocyte diversity. Similarly, injection of polyclonal IgG in JH−/− mice increased thymocyte TCR Vβ diversity by 7-fold, while injection of monoclonal IgG or ovalbumin did not (Table II). These results show that diverse immunoglobulin promotes TCR Vβ diversity. In agreement with this function for immunoglobulin in promoting T cell diversity, μMT mice with serum immunoglobulin that is 4.8% of wild type, had 70 fold greater T cell diversity than JH−/− mice that had no serum immunoglobulin (Table I).

TABLE II

| | TCR diversity | Variation from |
|---|---|---|
| JH−/− recipient | of thymocytes | JH−/− |
| Not reconstituted | 6.5 × 10² | 1 |
| Reconstituted with Polyclonal IgG | 4.3 × 10³ | 6.6 |
| Reconstituted with Polyclonal B cells | 5.4 × 10³ | 8.3 |
| Reconstituted with Monoclonal IgG | 1.9 × 10¹ | 0.03 |
| Reconstituted with Monoclonal B cells | 5.3 × 10² | 0.8 |
| Reconstituted with Ovalbumin | 1.6 × 10² | 0.25 |

Since injection of polyclonal IgG alone increases TCR diversity, it was determined whether production of immunoglobulin was the mechanism by which adoptively transferred B cells promoted T cell diversity. To find out, serum immunoglobulin was quantified in mice recipients of wild type B cells, 4 weeks or later post-transfer. Table I shows that JH−/− mice with adoptively transferred B cells had no measurable serum immunoglobulin, while JH−/− mice injected with polyclonal IgG had levels of immunoglobulin in the serum that were 4.8 fold greater than wild type.

### EXAMPLE 7

### Polyclonal Immunoglobulin or Immunoglobulin Derived Products Increase T Cell Diversity of Thymocytes and Splenocytes

JH−/− and QM mice were injected intraperitoneally weekly from birth until 4 weeks of age with 250 μg of (1) mouse polyclonal immunoglobulin (Ig; purchased from Serotec, Oxford UK), (2) mouse polyclonal reduced Ig (reduction of Ig dissulfite bridges), or (3) polyclonal Fab obtained by papain digestion of mouse IgG (purchased from Jackson Immunoresearch). The reduced Ig and Fab fragments were purified by HPLC and analyzed by SDS-PAGE. The diversity of thymocytes and splenocytes was determined using methods similar to those described elsewhere (Ogle et al., Nucleic Acids Res., 31:e139 (2003) and Joao et al., J. Immunol., 172:4709-4716 (2004)).

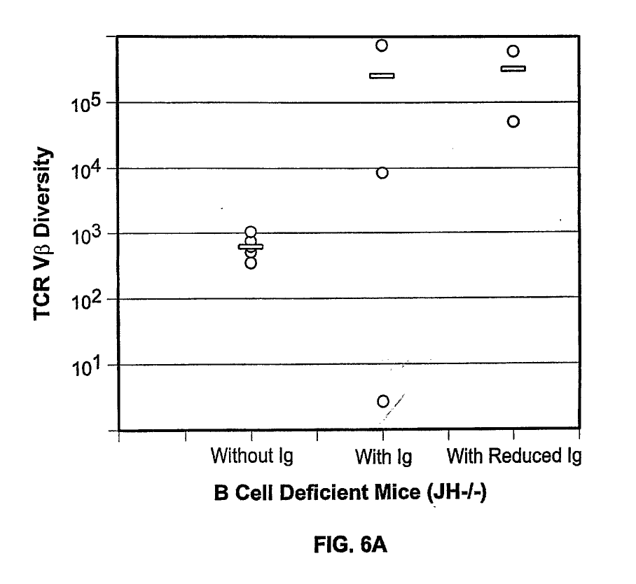

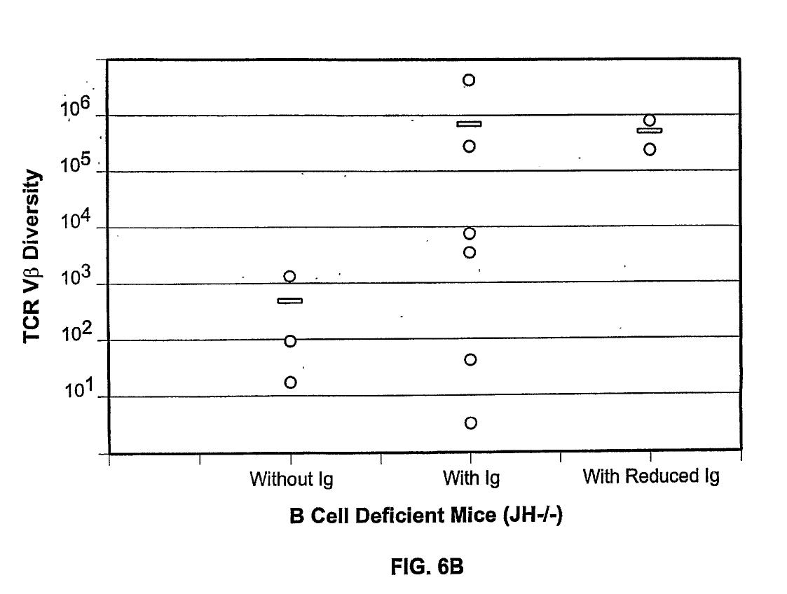

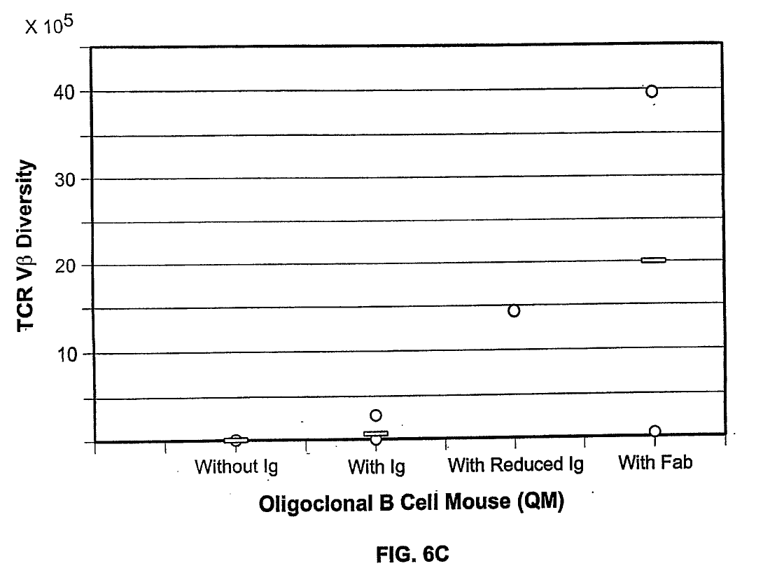

TCR Vβ diversity of thymocytes and splenocytes from JH−/− mice increased when the mice were treated with polyclonal Ig or polyclonal reduced Ig (FIGS. 6A and 6B). In addition, TCR Vβ diversity of splenocytes from QM mice increased when the mice were treated with polyclonal reduced Ig or polyclonal Fab (FIG. 6C).

### EXAMPLE 8

### Polyclonal Ig Increases Recent Thymic Emigrants

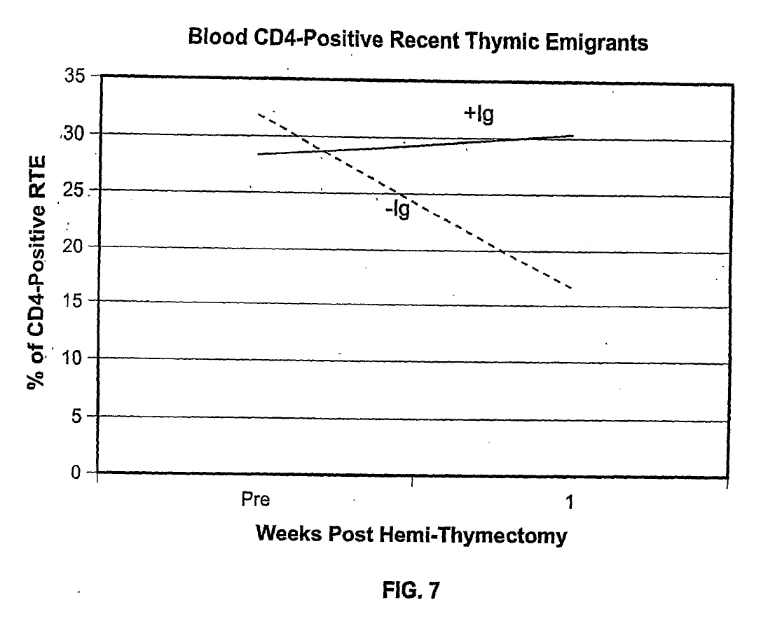

QM mice were hemi-thymectomized at 5 weeks of age and treated with weekly injections of 250 μg of mouse polyclonal Ig (purchased from Serotec, Oxford UK) immediately following hemi-thymectomy. Control mice were not treated with mouse polyclonal Ig. Recent thymic emigrants (RTE) were detected by BrdU incorporation as described elsewhere (Tough and Sprent et al., J. Exp. Med., 179:1127-1135 (1994) and Tough and Sprent, Curr. Protocols Immunol. Suppl., 18:4.7.1-4.7.6 (1996)). Treatment with polyclonal Ig increased RTE one week following hemi-thymectomy of QM mice (FIG.7).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for enhancing T cell diversity in a subject in need thereof, said method comprising administering a polyclonal population of B cells to said subject.

2. The method of claim 1, wherein said subject has an autoimmune disease.

3. The method of claim 1, wherein said autoimmune disease is selected from the group consisting of rheumatoid arthritis, insulin-dependent diabetes mellitus, myasthenia gravis, systemic lupus erythematosus, and inflammatory bowel disease.

4. The method of claim 1, wherein said subject has AIDS.

5. The method of claim 1, wherein said subject has a congenital immunodeficiency.

6. The method of claim 5, wherein said subject has severe combined immunodeficiency, common variable immunodeficiency, DiGeorge syndrome, or hyper IgM syndrome.

7. The method of claim 1, wherein said subject has cancer.

8. The method of claim 1, wherein said subject has a chronic infection.

9. The method of claim 1, wherein said subject has undergone partial or complete thymectomy.

10. The method of claim 1, wherein said subject is at least 20 years old.

11. The method of claim 1, said method further comprising monitoring T cell diversity in said subject.

12. The method of claim 11, wherein T cell diversity is monitored using a population of random or diverse nucleic acid molecules.

13. The method of claim 1, wherein said subject is a human.

14. A method for increasing T cell diversity in a subject in need thereof, said method comprising administering polyclonal immunoglobulin to said subject and monitoring T cell diversity in said subject.

15. The method of claim 14, wherein said subject has an autoimmune disease.

16. The method of claim 15, wherein said autoimmune disease is selected from the group consisting of rheumatoid arthritis, insulin-dependent diabetes mellitus, myasthenia gravis, systemic lupus erythematosus, and inflammatory bowel disease.

17. The method of claim 14, wherein said subject has AIDS.

18. The method of claim 14, wherein said subject has a congenital immunodeficiency.

19. The method of claim 18, wherein said subject has severe combined immunodeficiency, common variable immunodeficiency, DiGeorge syndrome, or hyper IgM syndrome.

20. The method of claim 14, wherein said subject has cancer.

21. The method of claim 14, wherein said subject has a chronic infection.

22. The method of claim 14, wherein said subject has undergone partial or complete thymectomy.

23. The method of claim 14, wherein said subject is at least 20 years old.

24. The method of claim 14, wherein said polyclonal immunoglobulins are Fab fragments.

25. The method of claim 14, wherein said polyclonal immunoglobulins are reduced monomers.

26. The method of claim 14, wherein said polyclonal immunoglobulin is recombinant.

27. The method of claim 14, wherein T cell diversity is monitored using a population of random or diverse nucleic acid molecules.

28. A method for enhancing T cell diversity in a thymectomized subject, said method comprising administering polyclonal immunoglobulin to said subject.

29. An article of manufacture comprising (a) polyclonal immunoglobulin or a polyclonal population of B cells and (b) packaging material indicating that said polyclonal immunoglobulin or said polyclonal population of B cells can be administered to a subject to increase T cell diversity.

30. The article of manufacture of claim 29, wherein said article of manufacture comprises a reagent for monitoring said T cell diversity.

31. The article of manufacture of claim 30, wherein said reagent is a nucleic acid molecule.

32. The article of manufacture of claim 29, wherein said article of manufacture comprises said polyclonal immunoglobulin.
